(19) United States
(12) Patent Application Publication
(10) Pub. No.: US 2014/0257048 A1
(43) Pub. Date: Sep. 11, 2014

(54) Omnisign medical device

(71) Applicant: Jassin Jouria, Hollywood, FL (US)

(72) Inventor: Jassin Jouria, Hollywood, FL (US)

(21) Appl. No.: 13/815,511

(22) Filed: Mar. 8, 2013

(51) Int. Cl. A61B 5/0205; G06F 19/00; A61B 5/00

(52) U.S. Cl. CPC A61B 5/0205; A61B 5/6802; G06F 19/322
USPC 600/301; 600/300; 705/3

(57) ABSTRACT

The OmniSign Medical Device is a hand held unit that senses, retrieves, and reveals all four main vital signs for a patient through an adjustable cuff portion that wraps around the patients arm and has three sensors to sense the blood pressure, pulse, temperature, and respiratory rate and reveals those results on the front screen and that information is transferred by WiFi to a main server for electronic storage and for easy access to that information in the future.

### BACKGROUND OF THE INVENTION

(1) Field of the Invention

The OmniSign Medical Device is a new invention in the field of the medical industry and profession as in being able to produce all four main vital signs opposed to one to three that is available now. This is a new utility application with a previous provisional application with No. 61/612,091. This new utility application has some changes to the design of the device but has maintained the functionality. The changes were made due to changes in the technology available and understood at the time of the provisional application. There will be more changes in this application through the engineering process.

(2) Description of the Art Including Information Disclosed under 37 CFR 1.97 & 1.98

The art described in the drawings of the design and utility function illustrate the unique ability of the OmniSign Medical Devise both in its use and its features that allow it to be used in many environments.

### BRIEF SUMMARY OF THE INVENTION

The OmniSign Medical Device is a new design with a new and unique functionality. It is able to detect and produce all four main vital signs at the same time through a specially designed software or firmware that in conjunction with the cuff around the patients arm is accurate and quick in its response.

### BRIEF DESCRIPTION OF THE SEVERAL VIEWS OF THE DRAWINGS

There are 5 drawings in total that describe and illustrate the design and function of the “OmniSign Medical Device”.

FIG. 1: Illustrates the front view main unit and cuff of the device

FIG. 2: Illustrates the front view of the main unit showing the results

FIG. 3: Illustrates the back side view of the main unit

FIG. 4: Illustrates the top view of the main unit

FIG. 5: Illustrates the front view of the cuff

### DETAILED DESCRIPTION OF THE PREFERRED EMBODIMENT

The OmniSign Medical Device drawings as described in the following:

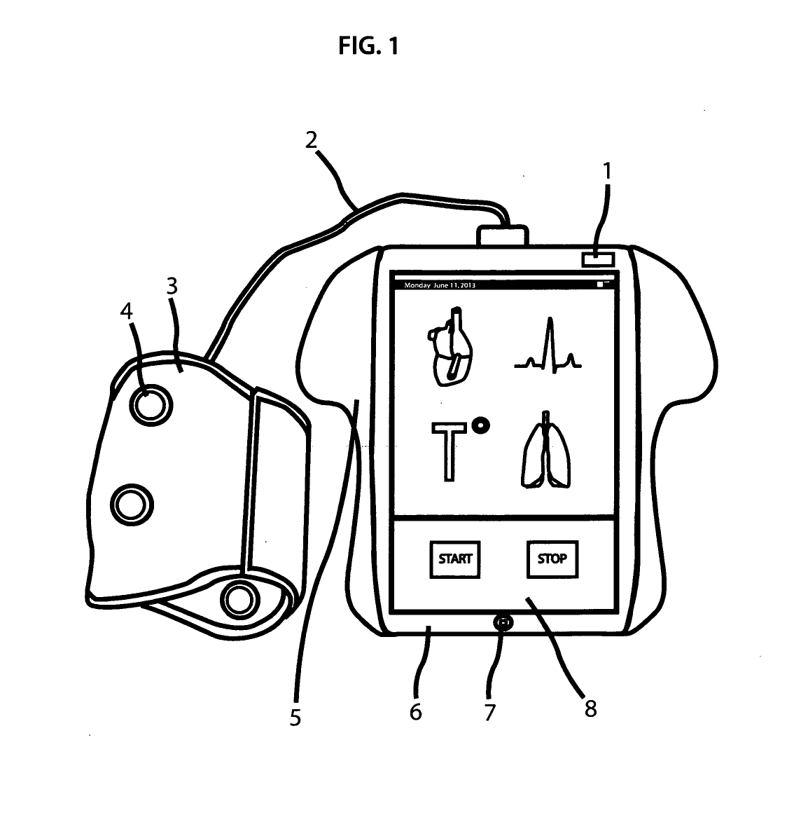

FIG. 1:

Illustrates the main unit and cuff with #1 the WiFi window to send data to a main frame server with #2 the cord connecting the cuff portion to the main unit with the USB plug in with #3 the body of the cuff portion with #4 the sensor for detecting the pulse and blood pressure of the patient with #5 the flange that the thumb slides under with #6 the body of the main unit with #7 the on/off power Button with #8 the face of the main unit showing the start and stop touch buttons with the four vital signs with the blood pressure vital sign on the top left and the pulse on the top right and the temperature on the bottom left and the respiratory rate on the bottom right.

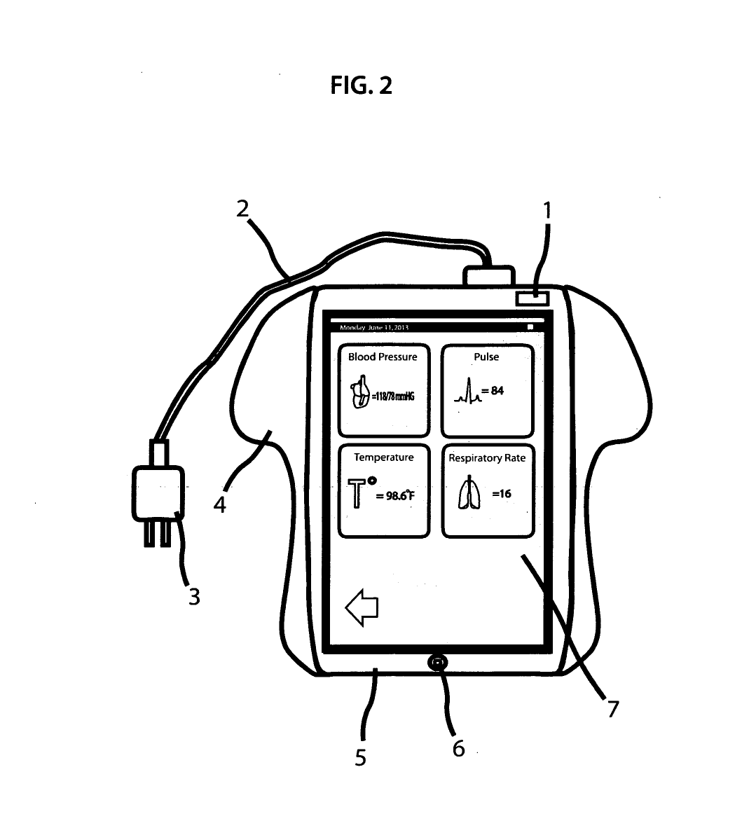

FIG. 2:

Illustrates the main unit with #1 the WiFi window for sending data to a main frame server with the cord of the charger with #3 the wall plug portion with #4 the flange allowing the thumb to slide into with #5 the main body of the unit with #6 the on/off power button with #7 the face of the main unit showing the results of the examination illustrating the blood pressure reading on the top left with the pulse reading on the top right with the temperature reading on the bottom left with the respiratory rate reading on the bottom right.

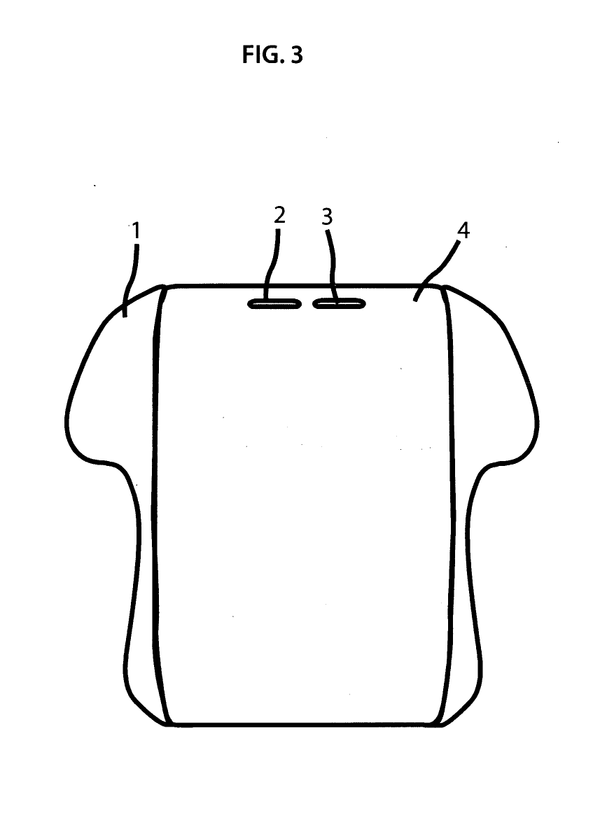

FIG. 3:

Illustrates the back side of the main unit with #1 the flange for the thumb and fingers to hold into for a non-slip feature with #2 the plug in for the wall charger with #3 the USB plug in for the cuff portion of the unit with #4 the main body of the main unit.

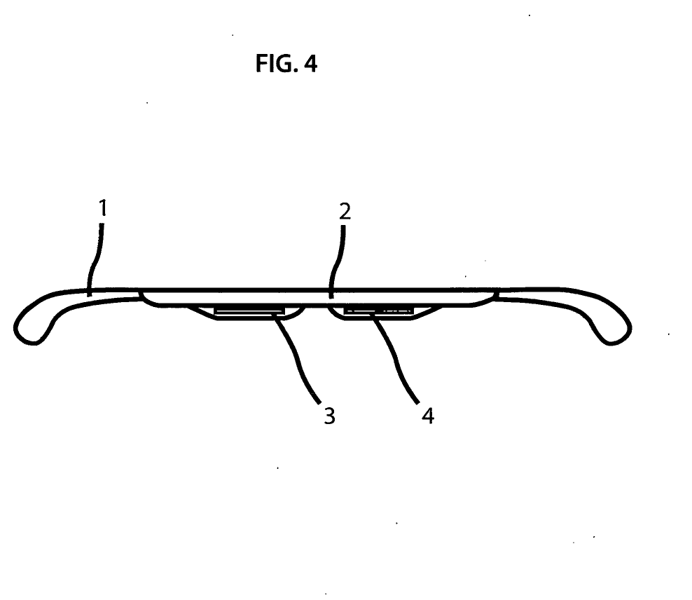

FIG. 4:

Illustrates the top view of the main unit showing the #1 flange with a downward angle to accommodate the thumbs or fingers to allow for a non-slip feature to the unit with #2 the main body of the main unit with #3 the plug in for the wall charger with #4 the USB plug in for the cuff portion of the unit.

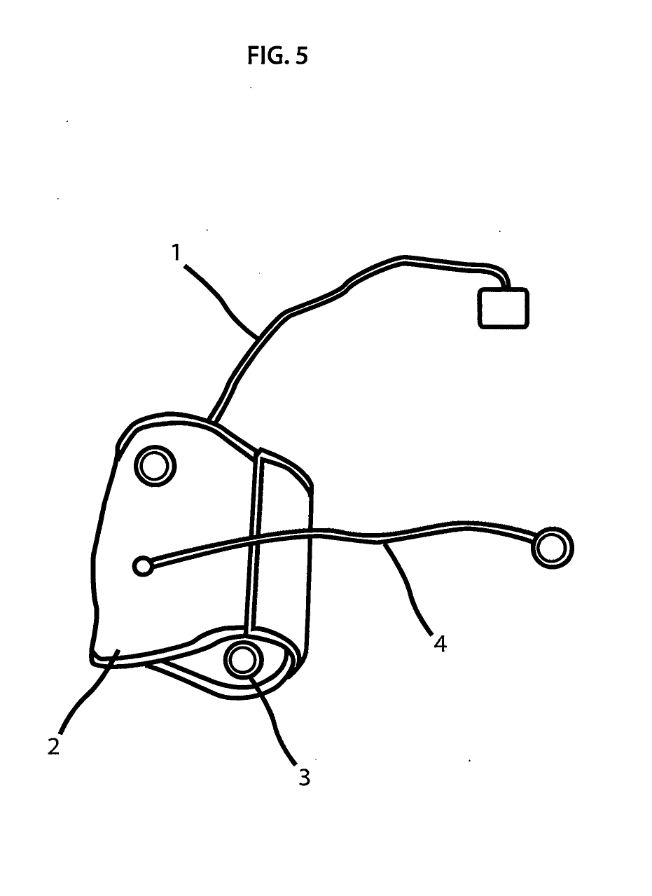

FIG. 5:

Illustrates the cuff portion of the unit with #1 the cord with the USB plug connected to the main unit with #2 the main body of the cuff portion with #3 the body temperature sensor with #4 the extendable respiratory rate sensor that can be stationary into the cuff portion or can be extended in specific situations for the patient.

### DETAILED DESCRIPTION OF THE INVENTION

The OmniSign Medical Device is a new design and functionality as to retrieving a patients four main vital signs within one application per patient. It uses the firmware technology as well as software functionality as the I pad, Apple products, Sony, Samsung and many others. The main unit is held in the hand of any professional doctor or professional practitioner and is the size of a typical I pad to be placed into the pocket for easy handling. The main unit can be charged the same way a cell phone is charged and is connected to a cuff portion that fits around a patients arm. Once the main unit is in start mode it will retrieve all four of the vital signs which are the blood pressure, the pulse, the temperature, and the respiratory rate using sensors built into the cuff itself. Using the cuff portion the respiratory rate sensor can be extended to be placed directly on the heart or side of the back or the back behind the heart area and or lung area. It has an LED or LCD screen that shows the vital sign results directly to the medical professional and the data can be transferred by WiFi to a main frame server or type available and the patients records are recorded and kept electronically. By being able to take all four vital signs at the same time and by using a device that can be placed into the professional pocket and the fact it is a very low cost to manufacture the low price point and the ease of use makes it very unique. The software or firmware is custom programmed and further information shall be provided as the device is further engineered to establish the specific code sourcing and code streams to better identify the devices uniqueness in its design and functionality.

What is claimed is:

1. The OmniSign medical device invention is a hand held medical device that retrieves all four of the main vital signs for a patient such as:
A. the blood pressure
B. the pulse
C. the temperature
D. the respiratory rate

2. The OmniSign medical device invention uses a software functionality to retrieve the vital signs through a cuff portion that wraps around the patients arm using sensors built into the cuff portion by:
A. the blood pressure and pulse sensor being one sensor with two functions.
B. the temperature sensor being one sensor with one function.
C. the respiratory sensor being one sensor having two functions being locked into the cuff and by having the ability to extend from the body of the cuff to be placed onto the patient directly.

3. The OmniSign medical device invention has a front screen that uses LED or LCD technology to display the four vital signs in square portions that are top left with top right with bottom left with bottom right to make the four squares to reveal the vital signs results.

4. The OmniSign medical device invention uses a cuff portion that plugs into the main unit which wraps around the patients arm which is adjustable to fit any arm size using specific sensors that touch specific parts of the human arm to sense readings and retrieving results for the main unit to show on the screen on the main unit.

5. The OmniSign medical device invention uses a WiFi technology that will transfer data results to a main frame server or type available to store a patient's medical records electronically and for easy retrieval from the main unit when that information is needed in the future.
